(19) United States
(12) Patent Application Publication
(10) Pub. No.: US 2009/0170865 A1
(43) Pub. Date: Jul. 2, 2009

(54) Treatment of Prostate Cancer with Angiogenesis-Targeting Quinazoline-Based Anti-Cancer Compounds

(75) Inventors: Natasha Kyprianou, Lexington, KY (US); Jason B. Garrison, San Diego, CA (US); Ching-Shih Chen, Columbus, OH (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 12/323,073

(22) Filed: Nov. 25, 2008

Related U.S. Application Data
(60) Provisional application No. 61/004,897, filed on Nov. 30, 2007.

(51) Int. Cl. A61K 31/517; C12N 5/06

(52) U.S. Cl. 514/252.17; 435/375

(57) ABSTRACT

Provided is a method of inhibiting the growth of prostate cancer cells comprising administering an effective amount of DZ-50 (2-[4-biphenyl-4-sulfonyl)-piperazin-1-yl]-6,7-diisopropoxyquinazolin-4-yl-amine) to a patient in need thereof. In another aspect, a method is provided for inhibiting the initiation of prostate cancer comprising administering an effective amount of DZ-50 to a patient in need thereof. In yet another aspect, a method is provided for inhibiting the formation of a prostate tumor-derived metastatic lesion comprising administering an effective amount of DZ-50 to a patient in need thereof. In any of the aforementioned methods, a quinazoline-based drug which induces apoptosis of a prostate cancer cell may be coadministered with DZ-50. Also provided is a composition comprising DZ-50, a quinazoline-based drug which induces apoptosis of a prostate cancer cell, and a pharmaceutically acceptable carrier.

### FIELD OF THE INVENTION

The invention relates to angiogenesis-targeting quinazoline-based anti-cancer compounds and their use in treating prostate cancer.

### BACKGROUND OF THE INVENTION

Prostate cancer is a major contributor to cancer mortality in American males causing the death of approximately 30,000 men in 2006 (Jemal et al., Cancer J. Clin., 56: 106-130, 2006). Therapeutic modalities such as radical prostatectomy and radiotherapy are considered curative for localized disease, yet no treatments for metastatic prostate cancer are available that significantly increases patient survival (Hill et al., Oncology Reports, 9: 1151-1156, 2002). Clinical and experimental evidence implicates two components as contributors towards the emergence of the androgen-independent phenotype: activation of survival (apoptosis suppression) pathways and increased tumor neovascularization (Garrison et al., Current Cancer Drug Targets, 4: 85-95, 2004; Weidner, Eur. J. Cancer, 32A: 2506-2011, 1996). Consequently, targeting of apoptotic players is of vital therapeutic significance since resistance to apoptosis is not only critical in conferring therapeutic failure to standard treatment strategies, but anoikis (cell death upon detachment from extracellular matrix) also plays an important role in angiogenesis and metastasis of malignant cells (Frisch et al., Cell. Biol., 124: 619-26, 1994; Rennebeck et al., Cancer Res., 65: 11230-11235, 2005).

Angiogenesis is critical in tumor progression and metastasis, since a functional vascular supply is required for the continued growth of solid tumors, and the spread of cancer cells (Folkman, Nat. Med., 21: 27-31, 1995). Small non-growing tumors may remain dormant for years and the angiogenic switch to aggressive metastatic phenotype, involves a change in the local equilibrium between factors inducing blood vessel formation and those inhibiting the process (Holmgren et al., Nat. Med., 1: 149-153, 1995; Ferrara et al., Nature, 438: 967-74, 2005). During angiogenesis cells are in a dynamic state, lacking firm attachment to the extracellular matrix, and exceedingly vulnerable to anoikis. Consequently, targeting tumor endothelial cell survival by triggering anoikis, may provide a molecular basis for novel therapeutic strategies for metastatic prostate cancer. Two classes of angiogenesis-targeting agents consequently emerge: those preventing the development of neovasculature of tumors, (via inducing apoptosis and/or inhibiting cell proliferation and migration), and those that directly target the existing tumor vasculature (via anoikis of tumor endothelial and epithelial cells) (Dameron et al., Science, 265: 1582-1584, 1994; Horsman et al., Cancer Res., 66: 11520-11539, 2006).

The quinazoline-based compounds doxazosin and terazosin are known α₁-adrenoreceptor antagonists, clinically effective for the relief of benign prostate hyperplasia (BPH) symptoms via their ability to selectively antagonize the α₁ₐ-adrenoreceptors, distributed in the bladder neck and prostate gland (Kirby et al., Br. J. Urol., 80: 521-532, 1997). Recent experimental and clinical evidence however, documented additional antigrowth effects by the quinazoline-based adrenoceptor antagonists, via induction of prostate epithelial and smooth muscle cell apoptosis as one of the molecular mechanisms contributing to their overall long-term clinical efficacy in BPH patients (Kyprianou, J Urol., 169: 1520-1525, 2003; Chon et al., J Urol., 161: 2002-2008, 1999). Suppression of prostate tumor growth by these drugs proceeds via an α₁-adrenoceptor-independent mechanism, mediated by TGF-β1 apoptotic signaling (Partin et al., Br. J. Urol., 88: 1615-1621, 2003; Benning et al., Cancer Res., 62: 597-602, 2002), receptor-mediated apoptosis involving DISC formation and caspase-8 activity (Garrison et al., Cancer Res., 66: 464-472, 2006) and inhibition of Akt activation (Garrison et al., 2006; Shaw et al., J. Med. Chem., 47: 4453-4462, 2004).

The separation of doxazosin's effect on cancer cell apoptosis from its original pharmacological activity in vascular cells provides an intriguing molecular basis to develop a novel class of apoptosis-inducing agents through lead optimization. Our recent pharmacological exploitation of doxazosin's quinazoline nucleus led to the development of novel compounds with and without the characteristic “classic” apoptotic activity, but exhibiting potent anti-vascular activity (Shaw et al., 2004). In this study, we report the targeting, by the new lead quinazoline-based compounds, of prostate tumor epithelial and endothelial cell survival, migration, neovascularization and angiogenesis in vitro and in vivo.

### SUMMARY OF THE INVENTION

In one embodiment, a method is provided for inhibiting the growth of prostate cancer cells comprising administering an effective amount of DZ-50 (2-[4-biphenyl-4-sulfonyl)-piperazin-1-yl]-6,7-diisopropoxyquinazolin-4-yl-amine) to a patient in need thereof.

In another embodiment, a method is provided for inhibiting the initiation of prostate cancer comprising administering an effective amount of DZ-50 to a patient in need thereof.

In yet another embodiment, a method is provided for inhibiting the formation of a prostate tumor-derived metastatic lesion comprising administering an effective amount of DZ-50 to a patient in need thereof.

In any of the aforementioned methods, a quinazoline-based drug which induces apoptosis of a prostate cancer cell may be coadministered with DZ-50.

Still another embodiment provides a composition comprising DZ-50, a quinazoline-based drug which induces apoptosis of a prostate cancer cell, and a pharmaceutically acceptable carrier.

Other methods, features and advantages of the present invention will be or become apparent to one with skill in the art upon examination of the following detailed descriptions. It is intended that all such additional methods, features and advantages be included within this description, be within the scope of the present invention, and be protected by the accompanying claims.

### BRIEF DESCRIPTION OF THE DRAWINGS

FIG. 1 illustrates the effect of the quinazoline-derived compound DZ-50 on human prostate cancer cells. FIG. 1A shows the chemical structure of DZ-50: the 2,3-dihydro-benzo[1,4]dioxane-carbonyl moiety of doxazosin was replaced with the biphenyl aryl sulfonyl substituent, whereas the methoxy side chains were replaced with isopropyl propoxy functions. FIG. 1B shows apoptosis induction by quinazoline compounds. PC-3 cells were treated (10 μmol/L) for 24 h and apoptosis was measured by Hoechst staining. FIG. 1C shows apoptosis induciton by DZ-3. Fluorescence-activated cell sorting analysis of propidium iodide and bromodeoxyuridine staining was done on PC-3 cells treated with DZ-3 (10 μmol/L) and a negative control, DZ-50 (10 μmol/L). FIG. 3D shows cell death following DZ-50 treatment. Cell death was evaluated in endothelial and epithelial cell lines following 24 and 48 h (inset) of treatment with DZ-50 (5, 10, 15, 20, and 25 μmol/L) as described in Materials and Methods.

FIG. 2 illustrates that DZ-50 prevents cell migration and adhesion to ECM of human prostate tumor epithelial cells and vascular endothelial cells. FIG. 2A shows wounding assays performed on endothelial and epithelial cells, with the number of migratory cells quantified as described in Materials and Methods. There was a significant reduction in the migratory capacity detected in the vascular endothelial and tumor epithelial cells analyzed (*, P<0.0001; **, P<0.001; ***, P=0.004). FIGS. 2B and 2C show that DZ-50 partially inhibits prostate tumor epithelial cell attachment to ECM components. The ability of prostate cancer cells PC-3 to adhere to ECM protein components was evaluated after exposure to DZ-50 for 6, 9, and 12 h at concentrations of 5 and 10 μmol/L. Attached prostate cancer cells were counted on fibronectin- or collagen-coated culture dishes (columns, mean; bars, SD). DZ-50 reduced the ability of PC-3 cells to attach to either fibronectin or collagen, but this effect was not statistically significant. FIGS. 2D-I and 2D-II show that DZ-50 prevents prostate cancer epithelial cell adhesion to endothelial cells. Transendothelial migration assays were done to assess the ability of PC-3 prostate cancer cells to attach and migrate through a monolayer of HMVEC-L following exposure to DZ-50. In Fig. D-I, PC-3 cells were stained with the lipohilic tracer Dil and were subsequently added to a confluent monolayer of HMVEC-L and exposed to DZ-50 for 3 and 9 h. DAPI staining identified the nuclei. Epithelial cell adhesion to the endothelial cell monolayer was prevented following 9 h of exposure to the drug (10 μmol/L). No death was detected within the first 24 h of treatment, indicating that blocking of transendothelial tumor migration was not due to drug-induced loss of cell viability (D-II).

FIG. 3 illustrates that DZ-50 prevents angiogenesis in vitro and in vivo. FIGS. 3A and 3B show that in vitro angiogenesis is blocked following exposure to DZ-50. Endothelial cells were seeded in Matrigel in the presence or absence of either DZ-50 or doxazosin at 10 μmol/L concentration and tube formation was visualized and quantified in the presence or absence of VEGF, as described in Materials and Methods. Control (top) shows HUVEC tube formation with decisive branch points whereas DZ-50 shows severely abrogated branch point formation. FIG. 3B shows quantitative analysis of the data; a significant reduction in tube formation is detected in the presence of DZ-50 compared with controls, whereas the quinazoline compound DZ-10 (no effect on cell viability—negative control) does not change the ability of HUVEC cells to form multibranched tubular networks. VEGF cannot reverse the antiangiogenic effect of DZ-50. FIGS. 3C and 3D show that in vivo angiogenesis is blocked by DZ-50. Chorioallantoic membrane assays were done in the presence or absence of DZ-50, as described in Materials and Methods, and the number of blood vessels was counted.

FIG. 4 illustrates that DZ-50 targets the integrin expression profile in human prostate cancer cells. FIG. 4A shows a comparison of integrin β₁ expression on PC-3 prostate cells following 12-h exposure to DZ-50 (10 μmol/L) or vehicle control (DMSO). FIG. 4B shows a comparison of integrin β₁ expression on DU-145 prostate cells following 12-h exposure to DZ-50 (10 μmol/L) or vehicle control (DMSO).

FIG. 5 illustrates suppression of primary tumor growth in the human prostate cancer xenograftr model by DZ-50. FIGS. 5A and 5B show that tumor volume of prostate xenografts is reduced following DZ-50 treatment. Following s.c. inoculation of nude mice (n=6 per group) with either PC-3 (A) or DU-145 (B) human prostate cancer cells, DZ-50 (100 and 200 mg/kg) was administered p.o. (via oral gavage) to tumor-bearing hosts for 14 d (subsequent to palpable tumor formation). Tumor volume was measured daily as described in Materials and Methods. DZ-50 treatment significantly suppressed prostate tumor volume compared with the vehicle control (P<0.001). FIG. 5C shows primary inhibition of androgen-independent human prostate tumor growth by DZ-50. To determine the ability of DZ-50 to interfere with prostate cancer development, nude mice were s.c. inoculated (n=6 per group) with PC-3 cells with concurrent exposure (p.o.) to DZ-50 (200 mg/kg) for 2 wk. FIG. 5D shows prostate cancer xenografts that were excised from DZ-50-treated and vehicle control tumor-bearing mice, paraffin embedded, and then tissue sections (6 μmol/L) were subjected to immunohistochemical analysis of apoptosis, cell proliferation, and tumor vascularity (A and B). The three images represent TUNEL staining for apoptosis, CD31 immunoreactivity for vascularity, and Ki67 expression for cell proliferation (magnification, ×400).

FIG. 6 illustrates inhibition of metastasis of human prostate cancer cells by DZ-50. In the experimental metastasis assay, nude mice (n=7 per group) were injected with prostate cancer cells PC-3 (2×10⁶) through the tail vein. DZ-50 treatment (200 mg/kg) was initiated at 10 d postinoculation for 21 d. Evaluation of the lungs (under dissecting microsope) revealed a significant reduction in the number of metastatic lesions to the lungs in the DZ-50-treated group compared with vehicle control mice; P<0.05. Arrows, metastatic foci on the lungs.

### DETAILED DESCRIPTION OF THE INVENTION

Before the present compositions and methods are described, it is to be understood that the invention is not limited to the particular methodologies, protocols, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless the context clearly dictates otherwise.

Unless defined otherwise, all technical and scientific terms used herein have the same meanings as commonly understood by one of ordinary skill in the art to which this invention belongs. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

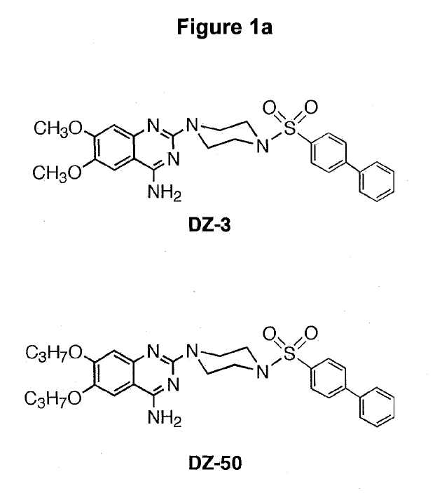

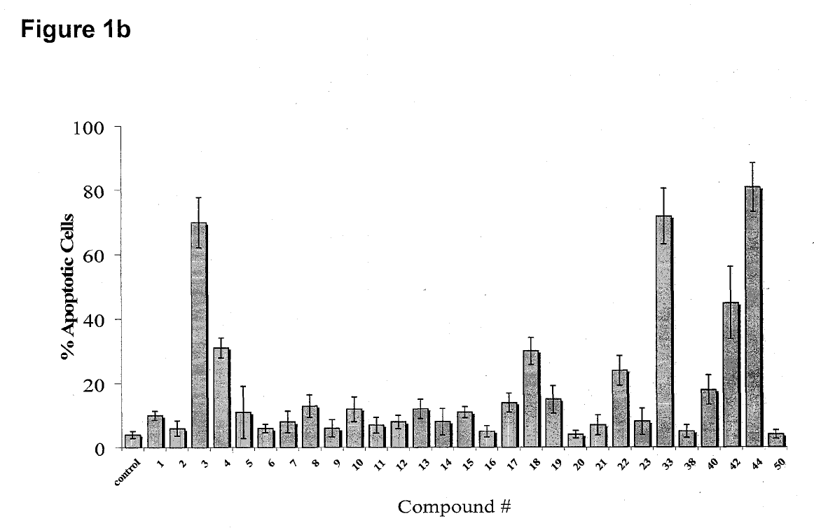

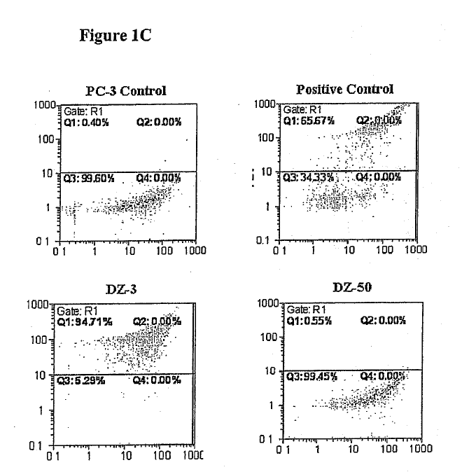

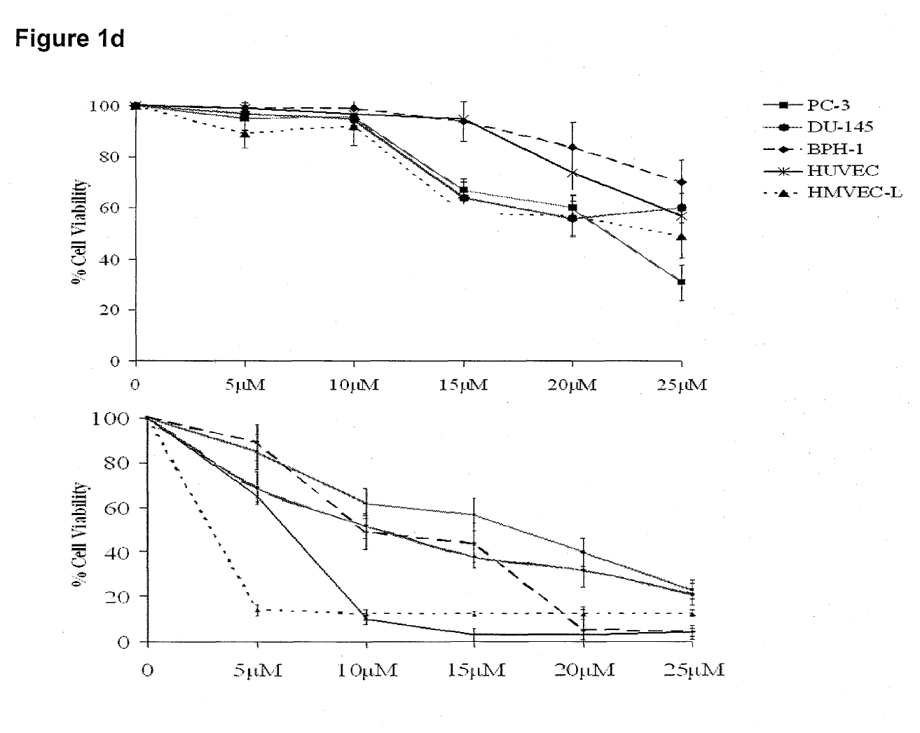

The present invention relates to the use of certain quinazoline-based drugs for the treatment of prostate cancer. In one aspect, the present invention is directed to the use of the quinazoline drug DZ-50 (see FIG. 1A) for the treatment of prostate cancer. DZ-50 has the chemical name 2-[4-biphenyl-4-sulfonyl)-piperazin-1-yl]-6,7-diisopropoxyquinazolin-4-yl-amine. DZ-50 induces a pattern of cell death that is independent of caspase-activation of apoptotic signaling. Rather, DZ-50 induces cell death by an anoikis mechanism. In particular, DZ-50 reduces the ability of prostate cancer cells to attach to the extracellular matrix and to migrate through endothelial cells. Hence, DZ-50 suppresses prostate cancer cell growth by targeting tissue vascularity.

Signaling pathways which are targeted by DZ-50 include the VEGF signaling pathway, the angiogenesis pathway, inflammation mediated by chemokine and cytokine signaling pathway, insulin/IGF pathway-mitogen activated protein kinase kinase/MAP kinase cascade, and the alpha adrenergic receptor signaling pathway.

In one aspect, the present invention is directed to a method of inhibiting the growth of prostate cancer cells comprising administering an effective amount of DZ-50 to a patient in need thereof.

In another aspect, the present invention is directed to a method of inhibiting the initiation of prostate cancer comprising administering an effective amount of DZ-50 to a patient in need thereof.

In yet another aspect, the present invention is directed to a method of inhibiting the formation of prostate tumor-derived metastatic lesions comprising administering an effective amount of DZ-50 to a patient in need thereof. The prostate tumor-derived metastatic lesions include those of the bone, lymph nodes, rectum, bladder and lung.

Any prostate cancer cell can be treated according to the present invention, including, human androgen-independent prostate cancer cells.

In another aspect of the present invention, prostate cancer cells are treated with DZ-50 in combination with another regimen for treating prostate cancer. The additional regimen can be administered at the same time as DZ-50, before treatment with DZ-50, or after treatment with DZ-50. The additional regimen of prostate cancer treatment is preferably one that is an apoptosis-inducing regiment, including the use of radiotherapy or administration of a quinazoline-based drug that induces apoptosis of prostate cancer cells. Thus, cotherapy with DZ-50 and the additional regiment affords attack of prostate cancer cells via both stimulating anoikis (via DZ-50) and stimulating apoptosis (via radiotherapy or an apoptosis-stimulating quinazoline-base drug such as DZ-3).

Examples of quinazoline-based drugs that induce apoptosis of prostate cancer cells include DZ-3 (2-[4-biphenyl-4-sulfonyl)-piperazin-1-yl]-6,7-dimethoxyquinazolin-4-yl-amine), DZ-44 (2-[4-biphenyl-4-sulfonyl)-piperazin-1-yl]-6,7-dipropoxyquinazolin-4-yl-amine), and DZ-42 (2-[4-(4′-tert-butylbiphenyl-4-sulfonyl)-piperazin-1-yl]-6,7-dimethoxyquinazolin-4-yl-amine). Additional examples of quinazoline-based drugs which can induce prostate cancer cell apoptosis are described in Shaw et al., J. Med. Chem., 47:4453-4462, 2004, the contents of which are hereby incorporated by reference. Any of the quinazoline-based drugs described herein, including DZ-50, DZ-3, DZ-44 and DZ-10 (2-[4-(2,4,6-triisopropylbenzenesulfonyl)-piperazin-1-yl]-6,7-dimethoxyquinazolin-4-yl-amine) can be made by the methods set forth in Shaw et al. Doxazosin and terazosin are additional quinazoline-based drugs which stimulate apoptosis of prostate cancer cells.

The compounds of the present invention may contain one or more stereocenters. The invention includes all possible diastereomers and all enantiomeric forms as well as all combinations of diasteriomers and enantiomers, including racemic mixtures. The compounds can be separated into substantially optically pure compounds.

The animals and cells treated according to the methods of the present invention preferably are mammals and mammalian cells. The methods can be used in any mammalian species, including human, monkey, cow, sheep, pig, goat, horse, mouse, rat, dog, cat, rabbit, guinea pig, hamster and horse. Humans are preferred.

The compounds of the present invention can be delivered directly or in pharmaceutical compositions along with suitable carriers or excipients, as is well known in the art. For example, a pharmaceutical composition of the invention may include a conventional additive, such as a stabilizer, buffer, salt, preservative, filler, flavor enancer and the like, as known to those skilled in the art. Exemplary buffers include phosphates, carbonates, citrates and the like. Exemplary preservatives include EDTA, EGTA, BHA, BHT and the like.

An effective amount of such agents can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences.

Suitable routes of administration may, for example, include oral, rectal, transmucosal, transdermal, topical, nasal, or intestinal administration and parenteral delivery, including intramuscular, subcutaneous, intramedullary injections, as well as intrathecal, direct intraventricular, intravenous, intraperitoneal, intranasal, or intraocular injections. In addition, the agent or composition thereof may be administered sublingually or via a spray, including a sublingual tablet or a sublingual spray. The agent or composition thereof may be administered in a local rather than a systemic manner. For example, a suitable agent can be delivered via injection or in a targeted drug delivery system, such as a depot or sustained release formulation.

The pharmaceutical compositions of the present invention may be manufactured by any of the methods well-known in the art, such as by conventional mixing, dissolving, granulating, dragee-making, levigating, emulsifying, encapsulating, entrapping, or lyophilizing processes. As noted above, the compositions of the present invention can include one or more physiologically acceptable carriers such as excipients and auxiliaries that facilitate processing of active molecules into preparations for pharmaceutical use.

Proper formulation is dependent upon the route of administration chosen. For injection, for example, the composition may be formulated in aqueous solutions, preferably in physiologically compatible buffers such as Hanks' solution, Ringer's solution, or physiological saline buffer. For transmucosal or nasal administration, penetrants appropriate to the barrier to be permeated are used in the formulation. Such penetrants are generally known in the art. In a preferred embodiment of the present invention, the present compounds are prepared in a formulation intended for oral administration. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use can be obtained as solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Also, wetting agents such as sodium dodecyl sulfate may be included.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In one embodiment, the compounds of the present invention can be administered transdermally, such as through a skin patch, or topically. In one aspect, the transdermal or topical formulations of the present invention can additionally comprise one or multiple penetration enhancers or other effectors, including agents that enhance migration of the delivered compound. Transdermal or topical administration could be preferred, for example, in situations in which location specific delivery is desired.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or any other suitable gas. In the case of a pressurized aerosol, the appropriate dosage unit may be determined by providing a valve to deliver a metered amount. Capsules and cartridges of, for example, gelatin, for use in an inhaler or insufflator may be formulated. These typically contain a powder mix of the compound and a suitable powder base such as lactose or starch.

Compositions formulated for parenteral administration by injection, e.g., by bolus injection or continuous infusion can be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents. Formulations for parenteral administration include aqueous solutions or other compositions in water-soluble form.

Suspensions of the active compounds may also be prepared as appropriate oily injection suspensions. Suitable lipophilic solvents or vehicles include fatty oils such as sesame oil and synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Alternatively, the active ingredient may be in powder form for constitution with a suitable vehicle, e.g., sterile pyrogen-free water, before use.

As mentioned above, the compositions of the present invention may also be formulated as a depot preparation. Such long acting formulations may be administered by implantation (for example, subcutaneously or intramuscularly) or by intramuscular injection. Thus, for example, the present compounds may be formulated with suitable polymeric or hydrophobic materials (for example as an emulsion in an acceptable oil) or ion exchange resins, or as sparingly soluble derivatives, for example, as a sparingly soluble salt.

Suitable carriers for the hydrophobic molecules of the invention are well known in the art and include co-solvent systems comprising, for example, benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system is effective in dissolving hydrophobic compounds and produces low toxicity upon systemic administration. Naturally, the proportions of a co-solvent system may be varied considerably without destroying its solubility and toxicity characteristics. Furthermore, the identity of the co-solvent components may be varied. For example, other low-toxicity nonpolar surfactants may be used instead of polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic molecules may be employed. Liposomes and emulsions are well known examples of delivery vehicles or carriers for hydrophobic drugs. Liposomal delivery systems are discussed above in the context of gene-delivery systems. Certain organic solvents such as dimethylsulfoxide also may be employed, although usually at the cost of greater toxicity. Additionally, the compounds may be delivered using sustained-release systems, such as semi-permeable matrices of solid hydrophobic polymers containing the effective amount of the composition to be administered. Various sustained-release materials are established and available to those of skill in the art. Sustained-release capsules may, depending on their chemical nature, release the compounds for a few weeks up to over 100 days. Depending on the chemical nature and the biological stability of the therapeutic reagent, additional strategies for stabilization may be employed.

For any composition used in the present methods of treatment, a therapeutically effective dose can be estimated initially using a variety of techniques well known in the art. For example, in a cell culture assay, a dose can be formulated in animal models to achieve a circulating concentration range that includes the IC₅₀ as determined in cell culture. Dosage ranges appropriate for human subjects can be determined, for example, using data obtained from cell culture assays and other animal studies.

A therapeutically effective dose of an agent refers to that amount of the agent that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are preferred.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods known in the art, in view of the specifics of a subject's condition.

The amount of agent or composition administered will, of course, be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein, and are specifically contemplated.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims.

### Materials and Methods

Cells Lines and Reagents The androgen-independent human prostate cancer PC-3 and DU-145 cell lines were obtained from the American Type Tissue Culture Collection (Rockville, Md.) and cultured in RPMI-1640 purchased from Invitrogen (Carlsbad, Calif.) containing 10% fetal bovine serum (Invitrogen) and antibiotics. The human benign prostatic epithelial cell line, BPH-1, [a gift from Dr. Simon W. Hayward (Department of Urological Surgery, Vanderbilt University Medical Center, Nashville Tenn.)] and were cultured in RPMI-1640 (Invitrogen) containing 10% fetal bovine serum and antibiotics. Human vascular endothelial cells (HUVEC) and human lung microvascular endothelial cells (HMVEC-L) were cultured in endothelial medium (EGM-2) (Cambrex, East Rutherford, N.J.) supplemented with EGM-2 and EGM2-MV (Cambrex). Recombinant human VEGF was purchased from Landing Biotech, (Newton, Mass.). Doxazosin derivatives (1-23, 38, 40, 42, and 50) were synthesized as described previously (Shaw et al., 2004).

### Apoptosis and Cell Viability Evaluation

a) Hoechst Staining. Cells were plated in 6-well culture dishes at 5×10⁴ cells per/well and at subconfluency were treated with increasing concentrations of DZ-1-23, -38, -40, -42, and -50 (0-25 μM). After 24 and 48 hrs of treatment, cells were fixed with 4% (w/v) paraformaldehyde (Sigma) and stained with 10 μg/mL Hoechst 33342 (B2261; Sigma) in the presence of 0.1% Triton X-100 (Sigma) as previously described. Cells were visualized using a Zeiss Axiovert S100 fluorescent microscope (Thornwood, N.Y.) with a UV filter (365 nm) and cells with condensed chromatin were designated apoptotic (100× magnification). The apoptotic index was determined by counting three random fields in duplicate wells per group. Each experiment was performed twice.
b) MTT assay: Subconfluent cultures of cells were exposed to increasing concentrations of DZ-1-23, -38, -40, -42, and -50 (0-25 μM). After treatment the medium was replaced with 250 μl of MTT (Sigma) (1 mg/ml) and incubated at 37° C. to form blue crystals. After 2 hrs the MTT was removed and replaced with DMSO (250 μl) and incubated overnight at 37° C. The DMSO-crystal solution's absorbance was read at 540 nm in a microplate reader (Bio-Tek Instruments, Winooski, VM). Numerical data represent the average of three independent experiments performed in triplicate.
Cell Migration Assay. (Wounding assay) Confluent monolayers of PC-3, DU-145, HUVEC, or HMVEC-L cells were wounded with a toothpick. After wounding, medium was changed and DZ-3 or DZ-50 (5 μM). After incubation for 12 or 24 hrs, wounding areas were examined under light microscopy (Axiovert 10, Zeiss). Cells that had migrated to the wounded areas were counted under a microscope for quantification of cell migration. Migration was calculated as the average number of cells observed in five random high power (400×) wounded fields/per well in duplicate wells.
Tube Formation Assay: In vitro Angiogenesis Evaluation: In vitro formation of tubular structures was studied on extracellular matrix using an angiogenesis kit as described by the manufacturer (Chemicon International, Inc., Temecula, Calif.). HUVEC or HMVEC-L (10×10⁴ cells/well) of 96-well-plates were seeded onto ECMatrigel-coated wells in the presence or absence of DZ-3 or DZ-50 and VEGF. Cells were treated with cytokines as single agents or each in combination (e.g. DZ-50 and VEGF). After 24 hrs post-treatment angiogenesis was assessed on the basis of formation of capillary-like structures of HUVEC, according to the manufacturer's protocol. The capillary-like tubes were counted (Nikon Eclipse, TE2000-U) in each well.

### Chicken Chorioallantoic Membrane (CAM) Assay

Fertilized chicken eggs were incubated at 37° C. At E8 a window was created to allow visualization of the egg shell membrane. 6 mm blank paper discs (BD) were placed on the egg shell membrane along with VEGF (100 ng) or bFGF (100 ng) and DZ-50. The windows were sealed with porous adhesive and allowed to incubate 48 hrs. At E10 the adhesive was removed along with the egg shell membrane to expose the CAM and 4% paraformaldehyde was added. Following excision the number of vessels per CAM was quantified by counting under a dissecting microscope.

### Cell Attachment Assay

Prostate cancer cells PC-3 and DU-145 cells were treated for 3, 6, 9, 12, or 15 hrs with DZ-50 (5 μM) and harvested. 5×10⁴ cells were added to each well of a 6-well culture dish coated with either collagen or fibronectin and incubated for 30 min at 37° C. Following incubation cells were fixed and the number of cells/well recorded. Numerical data represent the average of three independent experiments in triplicate.

### Transendothelial Migration (TEM) Assay

Sterile (12 mm diameter) glass coverslips were coated with Matrigel (Becton Dickson, Franklin Lakes, N.J.) at a dilution of 1:8 and air dried at room temperature (1 hr). Coverslips received approximately 6.25×10⁴ HMVEC-L to form a complete monolayer. The cells were allowed to spread on the Matrigel for 24 hrs prior to the experiment. PC-3 cells were resuspended in EGM-2MV (Cambrex) and added to the HMVEC-L monolayer at a concentration of 8×10³ cells/coverslip. Co-cultures were incubated at 37° C. at 5% CO₂ for 3, 6, 9, 12, and 24 hrs. Prior to the addition of prostate epithelial cells, cells were incubated with the lipophilic tracer 1,1′-dioctadecyl-3,3,3′,3′-tetramethylindocarbocyanine perchlorate (DiI) (Invitrogen, Carlsbad, Calif.) for 20 min at 10 μg/ml to stain cell membranes. To label F-actin, PC-3 cells, or co-cultures of PC-3 cells HMVEC-L were fixed for 10 min at room temperature in 2% paraformaldehyde in PBS, and were permeabilized for 5 min with a buffer containing 15 mM Tris, 120 mM NaCl, 2 mM EDTA, 2 mM EGTA, and 0.5% Triton X-100 (pH 7.4). Cells were incubated for 1 hr at room temperature with Alexam 488-conjugated phalloidin at a dilution of 1:50 in blocking solution, followed by 5 min of incubation with 10 mM Hoechst 33342 (Sigma) in PBS. Coverslips were mounted with Vectashield (Vector Laboratories, Burlington, Canada) on glass slides and analyzed with confocal microscopy.

### Western Blot Analysis

Cultures of PC-3, DU-145, BPH-1, HUVEC, and HMVEC-L cells were treated with DZ-50 (10 μM) for various time periods and cell lysates were subsequently generated in RIPA buffer [150 mM NaCl, 50 mM Tris pH 8.0, 0.5% deoxycholic acid, 1% Nonidet P40 with 1 mM phenyl methyl-sulfonyl fluoride (PMSF)]. The total protein concentration in the lysates was quantified by BCA Protein Assay Kit (Pierce, Rockford, Ill.) and protein samples (30 μg) were subjected to sodium dodecyl sulphate (SDS)-polyacrylamide gel electrophoresis, and transferred to Hybond-C membranes (Amersham Pharmacia Biotech., Piscataway, N.J.). After blocking with 5% dry milk in TBS-T (Tris-buffered-saline containing 0.05% Tween-20) for 1 hr (room temperature), membranes were incubated overnight at 4° C. with antibodies against caspase-8, Akt, or phosphorylated Akt (Cell Signaling Technology, Danvers, Mass.). Following incubation with the respective primary antibody, membranes were exposed to species-specific horseradish peroxidase (HRP)-labeled secondary antibodies. Signal detection was achieved with SuperSignal® West Dura Extended Duration Substrate (Pierce) and visualized using a UVP Bioimaging System (Upland, Calif.). All bands were normalized to α-actin expression (Oncogene Research Products™, La Jolla, Calif.).

FACS—Flow Cytometric Analysis: PC-3 cells were treated with DZ-50 (10 μM) and harvested with 0.5 mM EDTA solution. Prostate cancer epithelial cells were then incubated with hanks balanced salt solution (HBSS) supplemented with 2% BSA and 0.01% sodium azide for 30 min at 4° C. Cells were subsequently fixed in 4% (w/v) formaldehyde, washed, and incubated with the designated integrin antibody followed by FITC-conjugated goat anti-mouse secondary. Analysis was performed on a Partec FlowMax (Partec, Munster, Germany).

Tumorigenicity Studies. Human prostate cells (PC-3 and DU-145) suspended in PBS, were inoculated subcutaneously (s.c.) (2.5×10⁶ cells/site) in the flank of male nude mice, 4-6 weeks of age. Tumors were measured every 48 hrs with a digital caliber, and tumor volumes were calculated using the formula length×(width)²/2. When tumors reached ≈50 mm³ mice were stratified into treatment groups of 6 mice/treatment. DZ-50 was administered at doses of 50, 100, and 200 mg/kg in 0.5% methylcellulose (w/v)+0.1% Tween-80 (v/v) in water, by oral gavage using a 22-gauge, 1.5-inch gavage needle. Animals were sacrificed after 2 wks of treatment unless otherwise indicated. In a separate experiment human prostate cells (PC-3) were inoculated as described above and dosing began (200 mg/kg) concurrently for 2 wks. Upon termination of the experiment, tumors were surgically excised and tissue specimens were fixed in a 10% (v/v) formalin solution (Sigma) and subsequently embedded in Paraplast X-tra paraffin (VWR). Blocks were sectioned (6 μm) on a Finesse Microtome (ThermoShandon, UK).

Spontaneous Metastasis Assay: Human prostate cells (PC-3) were injected (2×10⁶ cells/80 μl of PBS) in the tail vein of male nude mice, 4-6 wks of age; mice were maintained in a pathogen-free environment. At 10 days post-inoculation, 200 mg/kg of DZ-50 was given daily (via oral gavage as described above). After 2 wks of treatment, DZ-50 treated and vehicle control mice were sacrificed and lungs, spleen, kidneys, and prostate organs were excised and subjected to examination for metastatic tumor lesions.
Apoptosis Evaluation Apoptotic cells were detected using the ApopTago Peroxidase In Situ Apoptosis Kit (Chemicon, Temecula, Calif.). Briefly, paraffin-embedded sections were treated with Proteinase K (Dako, Carpinteria, Calif.) and were subsequently incubated with terminal deoxynucleotidyl transferase enzyme. Terminal deoxynucleotidyl transferase-mediated deoxyuridine triphosphate nick end labeling (TUNEL)-positive cells were counted in five different fields (400×) and the apoptotic index was determined based on the number of apoptotic cells over the total number of cells.
Vascularity Evaluation: CD31 staining was performed for endothelial cells using enzymatic digestion with Proteinase K (Dako). The primary antibody used was the mouse anti-human CD31 specific for endothelial cells from Dako (overnight incubation at 4° C.). CD31-positive endothelial cells were counted in five different fields (400×).
Cell Proliferation Cell proliferation index was evaluated on the basis of Ki67 nuclear antigen immunoreactivity. Following antigen retrieval slides were incubated with an antibody directed against the Ki67 nuclear antigen (AMAC, Westbrook, Mass.). Ki67+ cells were counted from five different fields (400×).

### Statistical Analysis

One-way analysis of variance (ANOVA) was performed using the StatView statistical program to determine the statistical significance between values. A P value of less than 0.05 was considered statistically significant.

### Example 1

### DZ-50 is Effective at Inducing Cell Death Via a Non-Apoptotic Mechanism

Pharmacological exploitation of doxazosin's quinazoline nucleus led to the development of several novel agents with varying effects on apoptosis (FIG. 1A). Functional characterization of these compounds revealed two classes of agents: those that are not effective at inducing apoptosis, but elicit their effects by an alternative cell-death mechanism (DZ-50) and those that trigger apoptotic cell death (DZ-3) (FIG. 1A-1C). The most intriguing compound from the first category was DZ-50 that reduced cell viability in a number of endothelial and epithelial cells lines at both 24 and 48 hrs without induction of classic apoptosis (FIG. 1D).

### Example 2

### Anoikis Effect of DZ-50

### Inhibition of Cell Migration and Cell Adhesion

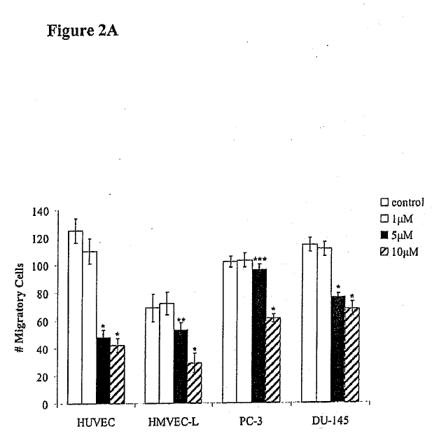

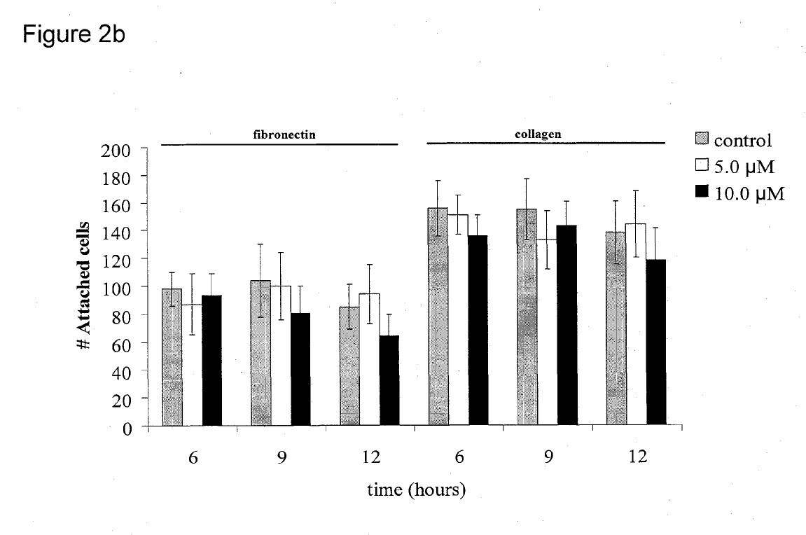

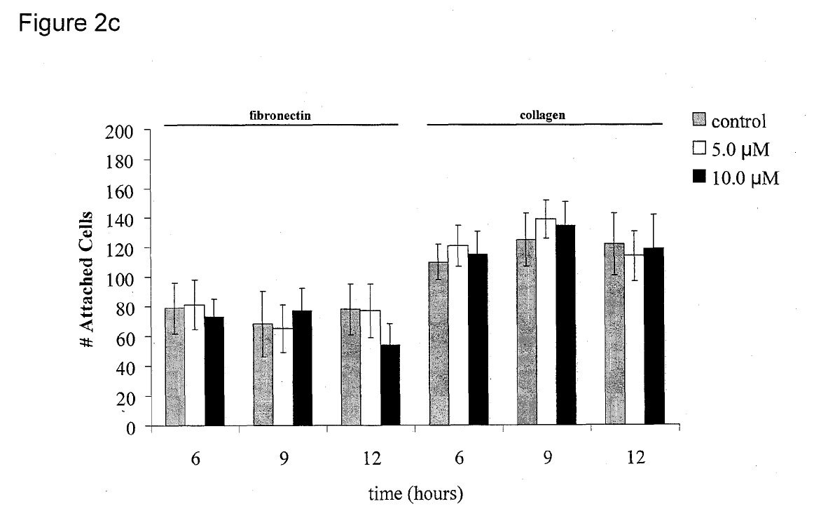

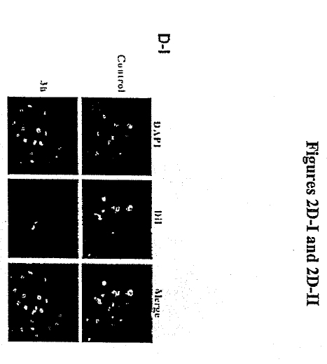

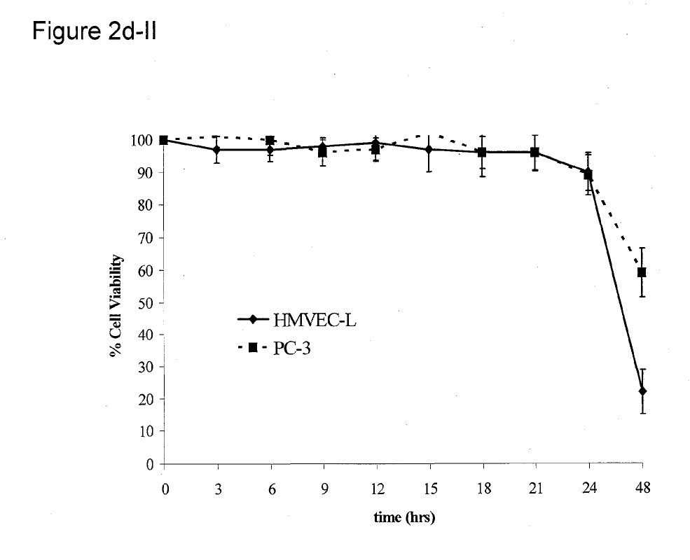

The ability of DZ-50 to potentially trigger anoikis of tumor epithelial and endothelial cells was subsequently investigated. Treatment with DZ-50 at (non-cytotoxic doses 5 and 10 μM) led to a significant inhibition of endothelial cell and prostate cancer epithelial cell (PC-3 and DU-145) migration (FIG. 2A). Moreover, exposure of PC-3 prostate cancer cells to DZ-50 reduced cellular adhesion to the extracellular matrix components fibronectin and collagen after 9-12 hrs (FIG. 2B), however this failed to reach statistical significance. Attachment of DU-145 prostate cancer cells to neither fibronectin nor collagen, was significantly inhibited by the drug treatment (FIG. 2C). Transendothelial migration assays were performed to assess the ability of PC-3 prostate cancer cells to migrate through an endothelial cell monolayer of HMVEC-L following exposure to DZ-50. PC-3 cells were stained with the lipophilic tracer DiI (red) and subsequently added to a confluent monolayer of HMVEC-L and exposed to DZ-50 for 3 and 9 hrs (FIG. 2D). DAPI staining identified the nuclei (blue). As shown on FIG. 2d, tumor epithelial cell adhesion to the endothelial cell monolayer was prevented following 9 hrs of exposure to the drug (10 μM). There was no effect on cell viability/cell death in either cell population (PC-3 nor HMVEC-L cells) in response to the drug DZ-50 (10M), with the first 24 hrs of treatment (FIG. 2D), indicating that the effect on transendothelial tumor cell migration was not due to drug-induced cell death.

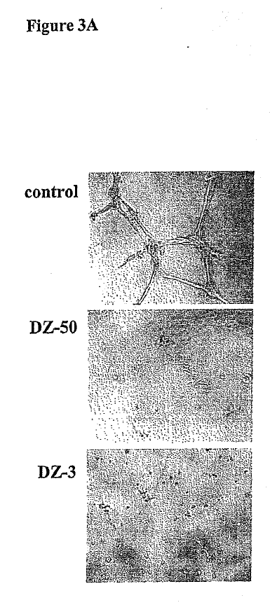

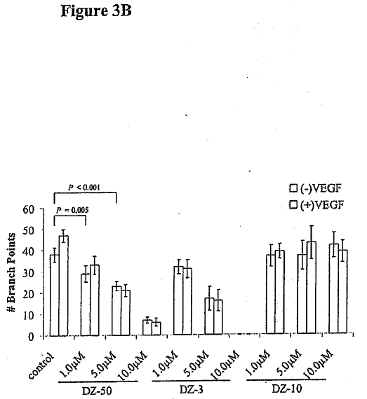

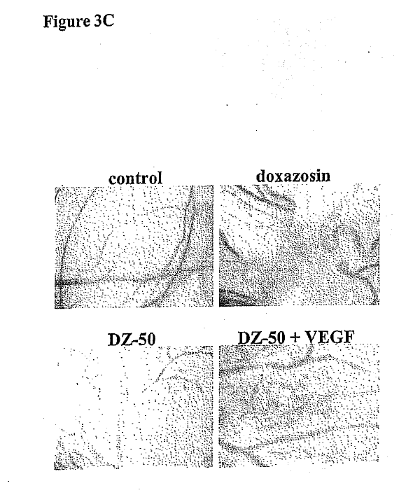

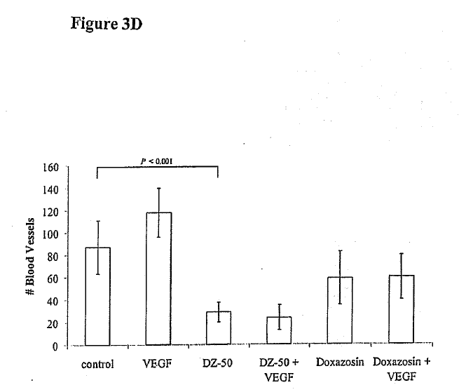

We subsequently investigated the direct effect of our lead drug DZ-50 on angiogenesis in vitro using the tube formation assay. As shown on FIG. 3 (panels A, B), following treatment with DZ-50, vascular endothelial cell tube formation was significantly inhibited. Furthermore, exposure to DZ-50 led to a significant suppression of angiogenesis/vascularity in the in vivo CAM blood vessel development assay (FIG. 3C, D). Simultaneous presence of a potent angiogenic factor VEGF and/or bFGF (data not shown) was not able to the rescue the cells from the antiangiogenic effect of DZ-50.

### Example 3

### Reduction of Integrin β1 Surface Expression by DZ-50

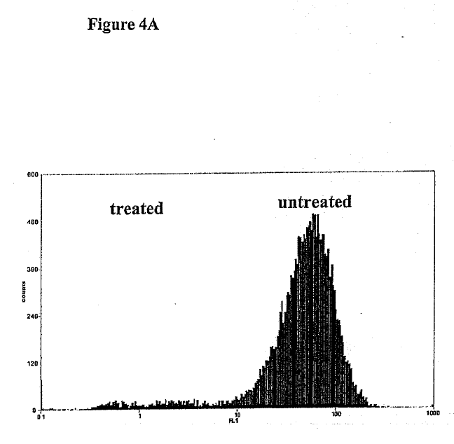

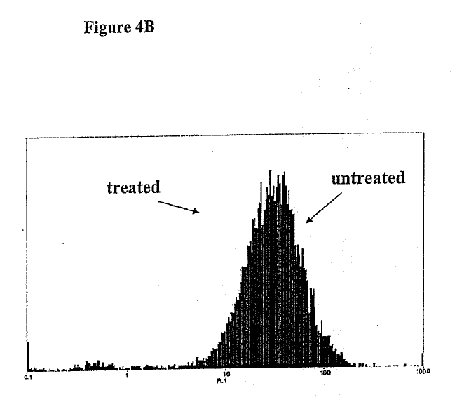

To explore the potential mechanism underlying that action of DZ-50 against prostate tumor epithelial cells, analysis of the integrin expression profile was performed. PC-3 untreated control cells were found to express integrin subunits α₂, α₃, α_{V}, β₁, and β₃. Exposure to DZ-50 did not effect the surface expression of integrins α₂, α₃, α_{V}, and β₃ (data not shown). As shown on FIG. 4 (panel A), integrin β₁ subunit was undetectable in cells treated with DZ-50 for 12-24 hrs, compared to vehicle control (FIG. 4A). DU-145 prostate cells exposed to DZ-50, exhibited a significantly smaller shift in integrin β₁ expression intensity (FIG. 4B).

### Example 4

### DZ-50 Treatment Suppresses Prostate Tumor Growth In Vivo

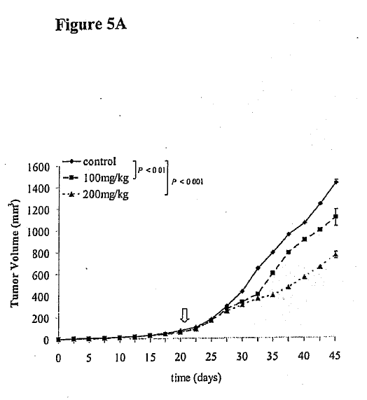

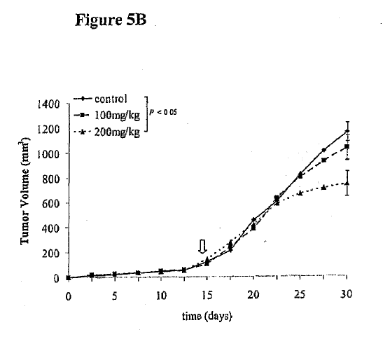

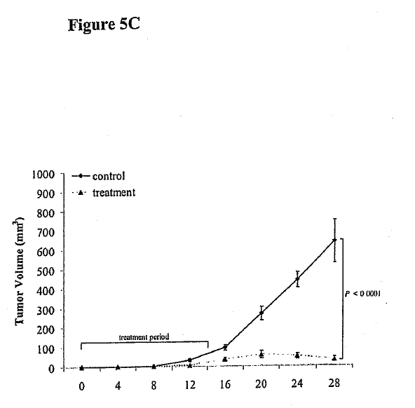

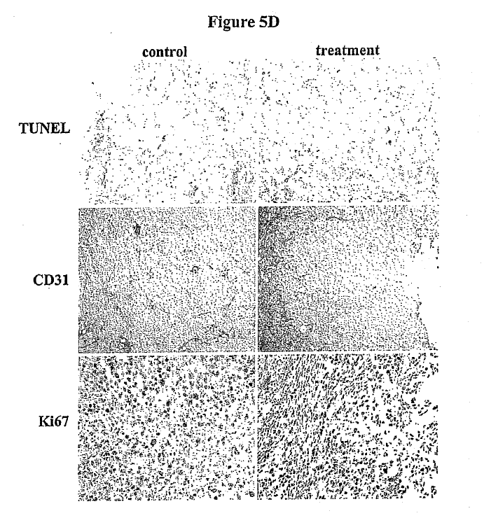

To assess the ability of DZ-50 to suppress prostate cancer growth we subsequently investigated the in vivo anti-tumor efficacy in human prostate cancer xenografts growing in nude mice. Our initial toxicity studies revealed no change in the animal's behavioral pattern and weight (data not shown). Both gross and histological examination of lung, liver, spleen, and prostate showed no apparent changes compared to control animals (data not shown). The tumorigenicity studies demonstrated a significant reduction in tumor volume in both androgen-independent human prostate cancer PC-3 and DU-145 tumor xenografts following treatment with DZ-50 (200 mg/kg) (FIG. 5A, B). The efficacy of DZ-50 to hinder the growth initiation of prostate tumors, was examined by inoculation of nude mice with PC-3 prostate cancer cells with simultaneous treatment with DZ-50 (200 mg/kg). As shown on FIG. 5 (panel C), prostate tumor development was dramatically suppressed with drug exposure (2 wks).

In situ detection of apoptosis in prostate tumors revealed no significant change in the apoptotic index of DZ-50 of prostate cancer xenografts from treated tumor-bearing mice compared to control (Table 1) further verifying that this compound does not induce apoptosis. Also shown on Table 1 is that there are no significant changes in the proliferative index of human prostate tumor xenografts from PC-3 and DU-145 cells derived from untreated and DZ-50 treated tumor bearing hosts. In contrast, treatment with DZ-50 led to a significant suppression of vascularity and angiogenesis, as detected by the reduced CD31 immunoreactivity in both PC-3 and DU-145 derived prostate tumor xenografts compared to the untreated prostate tissue (control mice) (Table 1). The results from the immunohistochemical analysis of prostate tumor apoptosis, vascularity and cell proliferation indicate that the DZ-50-mediated reduction in prostate tumor growth is, at least in part, consequential to targeting and reduction of angiogenesis.

TABLE 1

| | PC-3 | | | DU-145 | | |
|---|---|---|---|---|---|---|
| | Control | 100 mg/kg | 200 mg/kg | Control | 100 mg/kg | 200 mg/kg |
| TUNEL | 1.4 ± 0.3 | 2.0 ± 0.8 | 1.4 ± 0.4 | 3.2 ± 0.8 | 3.5 ± 1.0 | 3.4 ± 1.2 |
| (apoptotic | | | | | | |
| index) | | | | | | |
| CD31⁺ | 14.1 ± 0.8 | 13.5 ± 1.9 | 6.5 ± 0.6 | 18.5 ± 0.9 | 15.1 ± 0.7 | 10.1 ± 0.4 |
| (vascularity) | | | | | | |
| Ki67 | 43.7 | 42.6 | 45.0 | 51.2 | 53.9 | 49.7 |
| (proliferation | | | | | | |
| index), % | | | | | | |

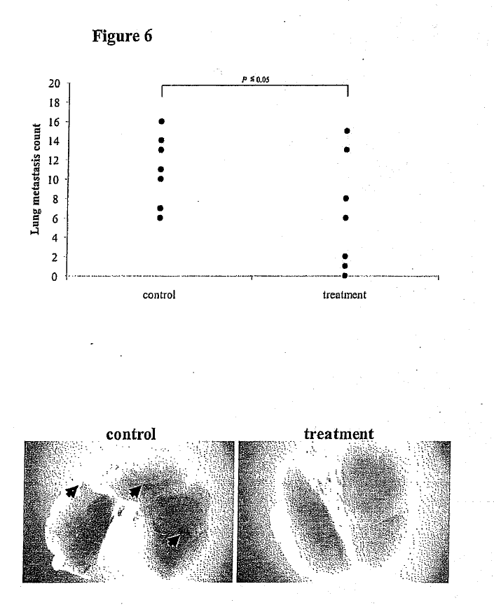

The ability of DZ-50 to directly affect tumor cell metastasis, was evaluated using the in vivo spontaneous metastasis assay. Following 21 days of DZ-50 treatment, there was a significant reduction in the number of metastatic foci to the lungs compared to the untreated control mice (FIG. 6). These results indicate the ability of DZ-50 to prevent and reduce prostate tumor growth, as well as inhibit invasion and metastatic potential in vivo.

This study demonstrates that DZ-50 effectively targets human prostate tumor epithelial cells as well as vascular endothelial cells, without inducing “classic” apoptosis. This unique feature of the anti-tumor action of the new drug, inducing a pattern of cell death that is independent of caspase-activation characteristic of apoptotic signaling, is mechanistically intriguing. The invasion process requires a range of cell-to-cell interactions, primarily through the association of adhesion complexes between tumor cells and the adjacent endothelial cells. The present findings indicate that DZ-50 triggers the anoikis phenomenon, as it interferes with prostate tumor cell migration and attachment to ECM components fibronectin and type I collagen (most abundant protein in bone). Examination of the ability of tumor cells to extravate by an in vitro model of transendothelial migration revealed that prostate tumor cells upon treatment with DZ-50, lost their ability to attach to the monolayer of endothelial cells; our results indicate that attachment of tumor epithelial cells to an endothelial monolayer was significantly inhibited after 6 hrs of exposure to DZ-50 and was completely abrogated after 9 hrs of treatment at non-cytotoxic doses. These in vitro data indicate that the lead compound can effectively minimize the possibility of transendothelial invasion and metastatic behavior of prostate cancer cells.

Collagen I binds the integrin pairs α₁β₁, α₂β₁, and α₃β₁ (Gullberg et al., EMBO J, 11: 3865-3873, 1992), and although we were unable to detect al expression in PC-3 and DU-145 prostate cells, there was strong expression of integrins α₂β₁ and α₃β₁. Following exposure to DZ-50, the PC-3 prostate cancer cells (originally isolated from a prostate tumor bone metastasis) exhibited complete loss of integrin β₁ surface expression, while the DU-145 prostate cancer cells had a minimal loss. Interestingly, human prostate cancer cells, characterized by a specific ability for bone metastasis, migrate toward collagen type I in an α₂β₁-dependent manner, leading to increased in vivo growth within the bone (Hall et al., Cancer Res., 66: 8648-8654, 2006). Thus one could argue that down regulation of integrin β₁ could provide the molecular basis for the response of prostate cancer cells to DZ-50. The regulation of β₁ integrin expression has been shown to be altered by TGF-β1 signaling (Cervella et al., J. Biol. Chem., 268: 5148-5155, 1993), at the transcriptional level by its attachment to the ECM and post-transcriptional/translational level (Delcommenne et al., J. Biol. Chem., 270: 26794-26801, 1995; Meleady et al., Cell Commun. Adh., 8: 45-59, 2001) and during differentiation (Hotchin et al., J. Biol. Chem., 267: 14852-14858, 1992) and cancer progression (Paulin et al., Leuk. Res., 25: 487-492, 2001). Moreover, integrin α₂β₁ mediates PC-3 cell adhesion to collagen and fibronectin, both major components of bone microenvironment (Gullberg et al., 1992), with some therapeutic promise. Thus, ionizing radiation leads to a significant reduction in β₁ integrin levels and decreasing cell adhesion to fibronectin (Simon et al., Prostate, 64: 83-91, 2005).

The present findings indicate that in vivo administration of the novel lead drug DZ-50 (at well-tolerated doses) not only significantly inhibits the growth of established human xenograft prostate tumors, but also prevents the initiation of prostate cancer development in this model. Moreover, exposure to DZ-50 resulted in a considerable suppression of the metastatic capacity of human prostate cancer cells, potentially by targeting their invasion and migration potential. Initial mechanistic dissection pointed to integrins as primary candidates of drug-targeting. Integrin β₁ knockout mice fail to develop a vasculature (Fassler, Genes Dev., 9: 1896-1908, 1995), so a direct functional link between reduced tumor growth and a lack of integrin β₁ is an attractive possibility. Furthermore, VEGF directly activates integrins α₅β₁ and α₂β₁, both implicated in angiogenesis (Byzova et al., Mol. Cell., 6: 851-860, 2000). One could easily argue that loss of integrin β₁ expression by DZ-50 (as detected in the present study), could interfere with VEGF signaling leading to reduced tumor vascularity, without affecting tumor cell death. VEGF has been specifically targeted by strategies such as monoclonal antibodies (bevacizumab) and inhibitors of endothelial cell receptor-associated tyrosine kinase activity (Ferrara et al., 2005). Other approaches including targeting basement membrane degradation, endothelial cell migration and endothelial cell proliferation have also been clinically evaluated, but success has been variable (Kerbel et al., Nat. Rev. Cancer, 2: 727-739, 2002; Eskens, Br. J. Cancer, 90:1-7, 2004).

Increases in patient survival in response to any antiangiogenic therapy have yet to be reported and current antiangiogenic therapy has been clinically ineffective. Phase III clinical trial data is lacking for any novel antiangiogenic compound; thus the immediate need for new targeted therapies for metastatic prostate cancer. Ongoing studies focus on dissecting the ability of the lead DZ compounds to target the interactions between integrin PI with its intracellular signaling partners. Decreased surface expression of integrin PI might result from down-regulation at the transcriptional or translational level. Alternatively, integrin β₁deregulation in response to DZ 50 might be an indirect effect from alterations in the focal adhesion complex [talin, focal adhesion kinase (FAK)], and other key components of the actin microfilaments that determine cell motility and migration. From a therapeutic standpoint either mechanism could prove beneficial, as by reducing the migratory capacity of tumor epithelial cells and/or inducing anoikis of endothelial cells, we could effectively prevent their ability to metastasize.

The observed effect of DZ-50 in preventing prostate tumor development in the xenograft model implies a prophylactic value for these compounds. Indirect support for such a concept stems from the recent epidemiological cohort study, indicating that exposure to doxazosin significantly decreases the incidence of prostate cancer among men (Harris et al., J Urol., 2007, November), thus suggesting a chemopreventive role for the quinazoline-based compounds. Finally, a combination of DZ-50 (targeting vascularity) with an apoptosis-inducing regimen for the treatment of metastatic prostate cancer emerges as an attractive therapy.

It will be appreciated that, although specific embodiments of the invention have been described herein for purposes of illustration, various modifications may be made without departing from the spirit and scope of the invention. All such modifications and variations are intended to be included herein within the scope of this disclosure and the present invention and protected by the following claims.

We claim:

1. A method of inhibiting the growth of prostate cancer cells comprising administering an effective amount of DZ-50 (2-[4-biphenyl-4-sulfonyl)-piperazin-1-yl]-6,7-diisopropoxyquinazolin-4-yl-amine) to a patient in need thereof.

2. The method of claim 1, wherein the prostate cancer cell is a human androgen-independent prostate cancer cell.

3. The method of claim 1, wherein a quinazoline-based drug which induces apoptosis of a prostate cancer cell is coadministered with DZ-50.

4. The method of claim 3, wherein the quinazoline-based drug is DZ-3 (2-[4-biphenyl-4-sulfonyl)-piperazin-1-yl]-6,7-dimethoxyquinazolin-4-yl-amine).

5. The method of claim 3, wherein the quinazoline-based drug which induces apoptosis of a prostate cancer cell is administered with DZ-50, before DZ-50, or after DZ-50.

6. A method of inhibiting the initiation of prostate cancer comprising administering an effective amount of DZ-50 to a patient in need thereof.

7. The method of claim 6, wherein the prostate cancer cell is a human androgen-independent prostate cancer cell.

8. The method of claim 6, wherein a quinazoline-based drug which induces apoptosis of a prostate cancer cell is coadministered with DZ-50.

9. The method of claim 8, wherein the quinazoline-based drug is DZ-3.

10. The method of claim 8, wherein the quinazoline-based drug which induces apoptosis of a prostate cancer cell is administered with DZ-50, before DZ-50, or after DZ-50.

11. A method of inhibiting the formation of a prostate tumor-derived metastatic lesion comprising administering an effective amount of DZ-50 to a patient in need thereof.

12. The method of claim 11, wherin the prostate cancer cell is a human androgen-independent prostate cancer cell.

13. The method of claim 11, wherein the metastatic lesion is inhibited from forming in the bone, lymph nodes, rectum, bladder or lung.

14. The method of claim 11, wherein a quinazoline-based drug which induces apoptosis of a prostate cancer cell is coadministered with DZ-50.

15. The method of claim 14, wherein the quinazoline-based drug is DZ-3.

16. The method of claim 14, wherein the quinazoline-based drug which induces apoptosis of a prostate cancer cell is administered with DZ-50, before DZ-50, or after DZ-50.

17. A composition comprising DZ-50, a quinazoline-based drug which induces apoptosis of a prostate cancer cell, and a pharmaceutically acceptable carrier.

18. The composition of claim 17, wherein the quinazoline-based drug is DZ-3.
